(19) United States
(12) Patent Application Publication
(10) Pub. No.: US 2022/0304607 A1
(43) Pub. Date: Sep. 29, 2022

(54) BIOLOGICAL INFORMATION MEASUREMENT SYSTEM AND COMPUTER-READABLE MEDIUM

(71) Applicants: Yoshihiro MISAKA, Ishikawa (JP); Hirofumi MORISE, Kanagawa (JP); Kiwamu KUDO, Ishikawa (JP)

(72) Inventors: Yoshihiro MISAKA, Ishikawa (JP); Hirofumi MORISE, Kanagawa (JP); Kiwamu KUDO, Ishikawa (JP)

(21) Appl. No.: 17/696,116

(22) Filed: Mar. 16, 2022

(30) Foreign Application Priority Data
Mar. 23, 2021 (JP) 2021-048825

(51) Int. Cl. A61B 5/245; G06T 7/73; A61B 5/055; A61B 5/369

(52) U.S. Cl. CPC A61B 5/245; G06T 7/74; A61B 5/055; A61B 5/369; G06T 2207/10012; G06T 2207/30196; G06T 2207/30016

(57) ABSTRACT

A biological information measurement system includes: a measurement unit configured to measure brain neural activity, based on biological signal detected by a plurality of sensors arranged in a cover member configured to cover a head of a subject; N image capturing units configured to each acquire an image including at least three reference points on the subject and the cover member; and a positional relationship determination unit configure to determine a positional relationship among a plurality of reference points on the subject and the plurality of sensors, based on positional relationship data among the plurality of reference points and the cover member, obtained based on N pieces of image data obtained from the image capturing units. An angle between image capturing directions of two image capturing units among the N image capturing units is larger than zero degree and smaller than 90 degrees.

### CROSS-REFERENCE TO RELATED APPLICATIONS

The present application claims priority under 35 U.S.C. § 119 to Japanese Patent Application No. 2021-048825, filed on Mar. 23, 2021. The contents of which are incorporated herein by reference in their entirety.

### BACKGROUND OF THE INVENTION

### 1. Field of the Invention

The present invention relates to a biological information measurement system and a computer-readable medium.

### 2. Description of the Related Art

Conventionally, in a magnetoencephalogram that measures magnetoencephalography (MEG), a weak biomagnetic field that is generated with human brain neural activity is measured and analyzed. The magnetoencephalogram as described above includes a dewar (housing) in which a number of magnetic sensors are incorporated. In the magnetoencephalogram, it is important to determine a positional relationship between the dewar and a head of a subject. In particular, detecting a head position of the subject using a camera or the like is desirable because it is possible to eliminate cumbersomeness, such as wearing of magnetic marker coils or the like, and because it is possible to perform detection in a non-contact manner.

For example, a technique for determining a positional relationship between the head and a biological activity measurement sensor by using a monocular camera (two-dimensional camera), a stereo camera (three-dimensional camera), or an image acquisition device that serves as a stereo camera in a pseudo manner using a monocular camera and a mirror and by using a pointer has been proposed.

As a method of making it possible to determine the positional relationship between the head of the subject and the dewar in real time and simultaneously measuring brain neural activity of the entire head, a method of constructing the dewar in a helmet form has been adopted. However, in this method, a reference point that is used to determine the position of the head is hidden depending on a position and an orientation of the head of the subject, and in some cases, it may be difficult to realize full coverage using only a single image capturing apparatus; therefore, accuracy in determination of the positional relation between the head and the biological activity measurement sensor may be reduced, which is a problem.

The present invention has been conceived in view of the foregoing situation, and an object of the present invention is to determine a positional relationship between a head of a subject and a sensor with high accuracy with respect to various positions and orientations of the head of the subject, and to improve reliability of analysis of biological information.

The conventional techniques are described in Japanese Unexamined Patent Application Publication No. 2020-054788, Japanese Unexamined Patent Application Publication No. 2020-168111, and Erich Urban et. al., “Optical Sensor Position Indicator for Neonatal MEG”, IEEE TRANSACTIONS ON BIOMEDICAL ENGINEERING, VOL. 59, NO. 1, JANUARY 2012, for example.

### SUMMARY OF THE INVENTION

According to an aspect of the present invention, a biological information measurement system includes a cover member, a measurement unit, N image capturing units, and a positional relationship determination unit. The cover member is configured to cover a head of a subject. A plurality of sensors configured to detect a biological signal are arranged in the cover member. The measurement unit is configured to measure brain neural activity, based on the biological signal detected by the plurality of sensors. The N image capturing units are configured to each acquire an image including at least three reference points and the cover member. The at least three reference points are set in relation to the subject. N is an integer equal to or larger than two. The positional relationship determination unit is configure to determine a positional relationship among a plurality of reference points on the subject and the plurality of sensors, based on positional relationship data among the plurality of reference points and the cover member. The positional relationship data is obtained based on N pieces of image data obtained from the image capturing units. An angle between image capturing directions of two image capturing units among the N image capturing units is larger than zero degree and smaller than 90 degrees.

### BRIEF DESCRIPTION OF THE DRAWINGS

FIG. 1 is a diagram illustrating an example of a system configuration of a biological information measurement system according to a first embodiment;

FIG. 2 is a diagram illustrating a situation in which reference points are hidden depending on an orientation of a head;

FIG. 3 is a diagram illustrating an example of a hardware configuration of an information processing device;

FIG. 4 is a diagram for explaining functions of the information processing device;

FIG. 5 is a diagram for explaining an overview of a method of determining a positional relationship;

FIG. 6 is a flowchart illustrating an example of the flow of a positional relationship determination process;

FIG. 7 is a diagram for explaining functions of an information processing device according to a second embodiment;

FIG. 8 is a diagram for explaining an example of a method of arranging image acquisition devices;

FIG. 9 is a diagram for explaining the example of the method of arranging the image acquisition devices;

FIG. 10 is a diagram for explaining the example of the method of arranging the image acquisition devices;

FIG. 11 is a graph illustrating an example of calculated values;

FIG. 12 is a diagram for explaining another example of the method of arranging the image acquisition devices;

FIG. 13 is a diagram for explaining the other example of the method of arranging the image acquisition devices; and

FIG. 14 is a diagram for explaining the other example of the method of arranging the image acquisition devices.

The accompanying drawings are intended to depict exemplary embodiments of the present invention and should not be interpreted to limit the scope thereof. Identical or similar reference numerals designate identical or similar components throughout the various drawings.

### DESCRIPTION OF THE EMBODIMENTS

The terminology used herein is for the purpose of describing particular embodiments only and is not intended to be limiting of the present invention.

As used herein, the singular forms “a”, “an” and “the” are intended to include the plural forms as well, unless the context clearly indicates otherwise.

In describing preferred embodiments illustrated in the drawings, specific terminology may be employed for the sake of clarity. However, the disclosure of this patent specification is not intended to be limited to the specific terminology so selected, and it is to be understood that each specific element includes all technical equivalents that have the same function, operate in a similar manner, and achieve a similar result.

An embodiment of the present invention will be described in detail below with reference to the drawings.

An embodiment has an object to determine a positional relationship between a head of a subject and a sensor with high accuracy for various positions and orientations of the head of the subject, and to improve reliability of analysis of biological information.

Embodiments of a biological information measurement system and a biological information measurement program will be described in detail below with reference to the accompanying drawings.

### First Embodiment

A biological information measurement system according to a first embodiment includes at least two image acquisition devices (image capturing units) that are arranged such that image capturing directions of the respective image acquisition devices are separated by a certain angle or more so as to capture a large number of reference points. With this configuration, even if a position and an orientation of the head are changed, it is possible to prevent reduction in accuracy of determination of a positional relationship between the head and a sensor due to hiding of the reference points.

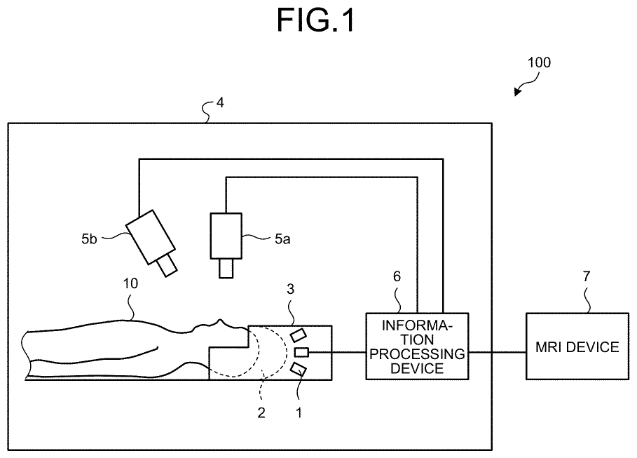

FIG. 1 is a diagram illustrating an example of a system configuration of a biological information measurement system 100 according to the first embodiment. As illustrated in FIG. 1, the biological information measurement system 100 includes a biological information measurement device 4 and a magnetic resonance imaging (MRI) device 7 that captures an MRI image. The biological information measurement device 4 includes a brain function measurement device 3, image acquisition devices 5a and 5b, and an information processing device 6.

The image acquisition devices 5a and 5b have the same functions, and therefore are simply referred to as the image acquisition devices 5 when they need not be distinguished from each other. While FIG. 1 illustrates an example in which the two image acquisition devices 5 are provided, embodiments are not limited to this example. The biological information measurement device 4 may be configured to include the N image acquisition devices 5 (N is an integer equal to or larger than two).

The brain function measurement device 3 is a magnetoencephalogram that measures a magnetoencephalography signal and an electro-encephalography (EEG) signal. A subject 10 as a measurement target inserts his/her head into a dewar 2 of the brain function measurement device 3 while wearing electroencephalography electrodes (or sensors) on the head, for example. The dewar 2 is a helmet type sensor-incorporated dewar serving as a cover member that surrounds almost the entire region of the head of the subject 10. The dewar 2 is a holding container in an extremely low temperature environment using liquid helium, and a large number of magnetic sensors 1 for magnetoencephalography are arranged inside the dewar 2. The brain function measurement device 3 collects an electro-encephalography signal from the electrodes and a magnetoencephalography signal from the magnetic sensors 1. The brain function measurement device 3 outputs the collected biological signals to the information processing device 6.

Meanwhile, the dewar 2 in which the magnetic sensors 1 are incorporated is generally arranged in a magnetic shielding room, but illustration of the magnetic shielding room is omitted for the sake of convenience in the illustration.

The information processing device 6 displays a waveform of the magneto-encephalography signal from the plurality of magnetic sensors 1 and a waveform of the electro-encephalography signal from the plurality of electrodes in a synchronized manner on the same time axis. The electro-encephalography signal represents electrical activity of a nerve cell (flow of ionic charges that occur at neuron dendrites during synapse transmission) as a potential difference between the electrodes. The magneto-encephalography signal represents small magnetic field variation that occurs due to brain electrical activity. A brain's magnetic field is detected by a superconducting quantum interference device (SQUID) sensor with high sensitivity.

Further, the information processing device 6 inputs a cross-sectional image (MRI image) of the head of the subject 10 captured by the MRI device 7. The MRI device 7 may capture the image before or after the brain function measurement device 3 performs magnetic measurement. Obtained image data is transmitted to the information processing device 6 by online or offline.

Meanwhile, as a cross-sectional image capturing device that captures a cross-sectional image of the head of the subject is not limited to the MRI device 7, but may be an X-ray computer tomography (CT) device or the like.

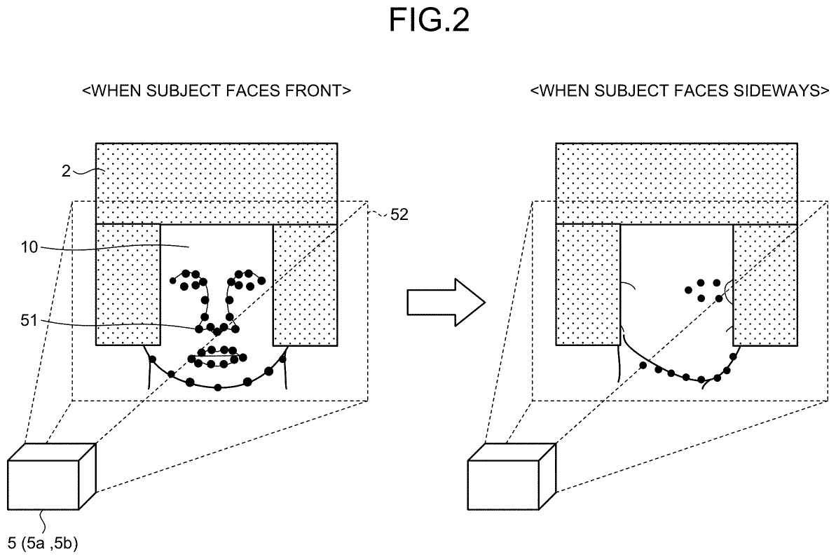

FIG. 2 is a diagram illustrating a situation in which reference points are hidden depending on an orientation of the head. In the left part and the right part in FIG. 2, a case in which the head of the subject 10 faces front (toward the image acquisition devices 5) and a case in which the head of the subject 10 faces sideways are illustrated. A plurality of reference points 51 are set on the head of the subject 10. As illustrated in FIG. 2, in an imaging range 52 of each of the image acquisition devices 5, the number of reference points 51 is larger in the case in which the head faces front than in the case in which the head faces sideways.

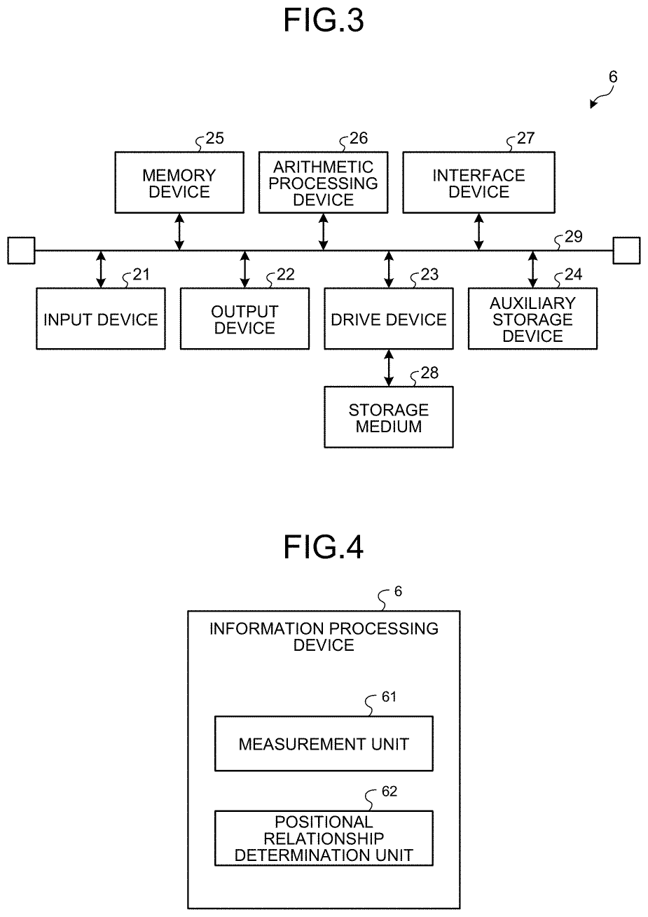

The information processing device 6 will be described in detail below. FIG. 3 is a diagram illustrating an example of a hardware configuration of the information processing device 6.

The information processing device 6 includes an input device 21, an output device 22, a drive device 23, an auxiliary storage device 24 for storing a biological information measurement program, a memory device 25, an arithmetic processing device 26, and an interface device 27, all of which are connected to one another via a bus 29.

The input device 21 is a device for inputting various kinds of information and implemented by, for example, a keyboard, a pointing device, and the like. The output device 22 is a device for outputting various kinds of information and implemented by, for example, a display or the like. The interface device 27 includes a local area network (LAN) card or the like and is used to connect to a network.

The biological information measurement program is at least a part of various programs for controlling the information processing device 6. The biological information measurement program is provided by, for example, distribution of a storage medium 28, download from the network, or the like. As the storage medium 28 in which the biological information measurement program is recorded, various types of storage media may be used; for example, a storage medium, such as a compact disk read only memory (CD-ROM), a flexible disk, or a magneto-optical disk, that optically, electrically, or magnetically records information, a semiconductor memory, such as a ROM or a flash memory, that electrically records information, and the like may be used.

Furthermore, if the storage medium 28 in which the biological information measurement program is recorded is set in the drive device 23, the biological information measurement program is installed in the auxiliary storage device 24 from the storage medium 28 via the drive device 23. The biological information measurement program that is downloaded from the network is installed in the auxiliary storage device 24 via the interface device 27.

The auxiliary storage device 24 stores therein the installed biological information measurement program, a necessary file, data, and the like. The memory device 25 reads the biological information measurement program from the auxiliary storage device 24 and stores therein the biological information measurement program when the information processing device 6 is activated. The arithmetic processing device 26 performs various processes as will be described later in accordance with the biological information measurement program stored in the memory device 25.

Meanwhile, it is assumed that the magnetic sensors 1 in the biological information measurement system 100 detect a signal that is generated by the brain neural activity of the subject 10, but it may be possible to use biological activity measurement sensors, such as optically pumped atomic magnetometers (OPAM). Further, it is assumed that the magnetic sensors 1 in the biological information measurement system 100 detect a signal that is generated by the brain neural activity of the subject 10, but embodiments are not limited to this example. It is sufficient that the biological information measurement system 100 includes a sensor (biological activity measurement sensor) for detecting a signal generated by the brain neural activity, and it is preferable that the sensor is less invasive, or more preferably, non-invasive, in order to accurately measure a biological function of the subject 10. Examples of the sensor as described above include a brain wave measurement sensor (potential sensor) and an optical topography (near-infrared sensor), in addition to the magnetic sensor.

Furthermore, the magnetic sensors 1 of the present embodiment may include a plurality of kinds of sensors. However, in this case, it is necessary to prevent operation of a single sensor from affecting measurement performed by a different sensor. In particular, if a magnetic sensor is used as one of the sensors, it is possible to acquire a signal generated by a living body even if the living body and the magnetic sensor do not contact with each other, and therefore, a mounting state of the sensor does not affect a measurement result. Therefore, the magnetic sensors 1 are preferable as an example.

The image acquisition devices 5 are, for example, image capturing units, such as monocular cameras or stereo cameras. It is sufficient that the image acquisition devices 5 are able to capture images of a range including the dewar 2 and the reference points. The image acquisition devices 5 each acquire an image including the reference points and the dewar 2 while the brain function measurement device 3 is measuring magnetoencephalography or the like. While details will be described later, the information processing device 6 is able to determine a positional relationship among the head of the subject 10 and the magnetic sensors 1 from the dewar 2, the reference points, and the like in the images. Therefore, the information processing device 6 is able to re-determine the positional relationship even if the head of the subject 10 moves while the brain function measurement device 3 is measuring magnetoencephalography or the like. According to the present embodiment, it is possible to determine the positional relationship between the head of the subject 10 and the dewar 2 in real time and simultaneously measure the brain neural activity of the entire head.

A functional configuration example of the information processing device 6 according to the present embodiment will be described below. FIG. 4 is a diagram for explaining functions of the information processing device 6.

The information processing device 6 includes a measurement unit 61 and a positional relationship determination unit 62.

The measurement unit 61 and the positional relationship determination unit 62 are implemented by causing the arithmetic processing device 26 to read the biological information measurement program stored in the auxiliary storage device 24, the memory device 25, and the like and execute the biological information measurement program.

The measurement unit 61 measures the brain neural activity on the basis of a biological signal (magnetoencephalography signal) that is detected in accordance with a stimulus.

The positional relationship determination unit 62 makes an association of the positional relationship among the plurality of reference points and the dewar 2, and determines the positional relationship among the head of the subject 10 and the magnetic sensors 1 in a three-dimensional space on the basis of structural data of the dewar 2 related to positions and postures of the magnetic sensors 1. In the present embodiment, the structural data of the dewar 2 is three-dimensional arrangement data of the magnetic sensors 1.

Further, the positional relationship determination unit 62 detects a change in the position of the head of the subject 10 on the basis of images that are captured by the image acquisition devices 5 (image capturing units) at different times, and re-determines the positional relationship among the reference points on the subject 10 and the magnetic sensors 1. For example, by overlapping first reference point group data of the subject 10 obtained at a first time and a second reference point group data of the subject 10 obtained at a second time, it is possible to calculate a change in the position of the head and re-determine the positional relationship with the magnetic sensors 1 on the basis of the change.

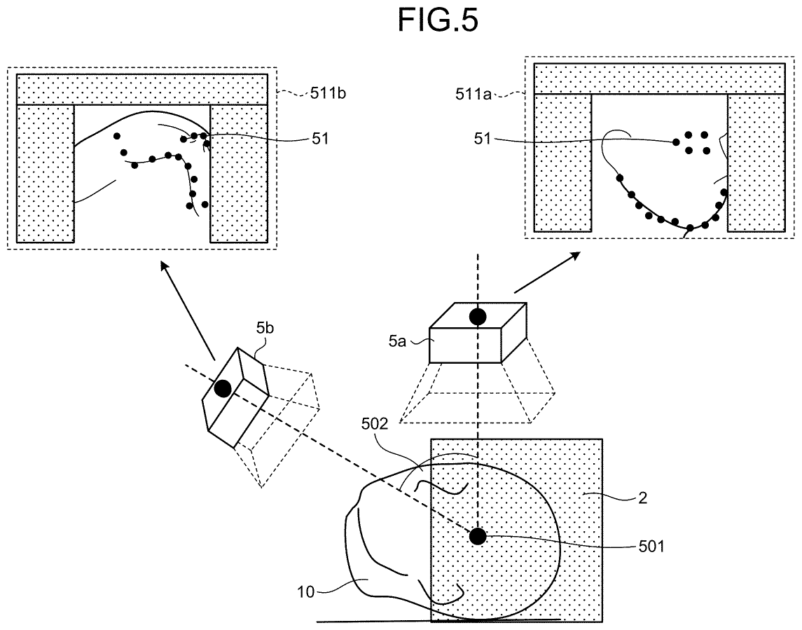

FIG. 5 is a diagram for explaining on overview of a method of determining the positional relationship. As described above, the reference points 51 are set on a surface of the head of the subject 10. The number of the reference points 51 to be set and setting positions may be selected arbitrarily as long as the reference points 51 are located on the surface of the head.

The information processing device 6 (for example, the memory device 25) stores therein, in advance, three-dimensional data representing a shape of the head of the subject 10 (hereinafter, the data will be referred to as three-dimensional head data). In other words, even if any point on the surface of the head is selected as the reference point 51, a three-dimensional (XYZ) coordinate of the reference point 51 is known on a coordinate system of the three-dimensional head data.

The image acquisition devices 5a and 5b are arranged such that the respective image capturing directions face a center 501 of the dewar 2. For example, if an angle 502 between the image capturing direction of the image acquisition device 5a and the image capturing direction of the image acquisition device 5b is denoted by θ, the image acquisition devices 5a and 5b are arranged such that θ is equal to or larger than a certain angle.

With use of the image acquisition devices 5a and 5b, each of an image 511a and an image 511b of the dewar 2 including the head of the subject 10 is captured. The positional relationship determination unit 62 extracts coordinates (x, y) of the reference points 51 on an image coordinate system in each of the image 511a and the image 511b.

The positional relationship determination unit 62 associates, in the image 511a obtained by the image acquisition device 5a, XY coordinates (x1, y1) of the reference points 51 that are extracted on the image coordinate system and XYZ coordinates of the reference points 51 that are present in the coordinate system of the three-dimensional head data.

Similarly, the positional relationship determination unit 62 associates, in the image 511b obtained by the image acquisition device 5b, XY coordinates (x2, y2) of the reference points 51 that are extracted on the image coordinate system and the XYZ coordinates of the reference points 51 that are present on the coordinate system of the three-dimensional head data.

Through the process as descried above, the positional relationship determination unit 62 is able to determine a positional relationship among the image acquisition devices 5 (5a and 5b) and the head of the subject 10.

A method of determining the positional relationship among the image acquisition devices 5 and the dewar 2 will be described below. Each of the image acquisition devices 5a and 5b is arranged at a certain position at which the image acquisition devices 5a and 5b are able to capture images of the dewar 2.

A process of determining the positional relationship among the image acquisition devices 5 and the dewar 2 is performed in the same manner as the process adopted in the method of determining the positional relationship among the image acquisition devices 5 and the subject 10. It may be possible to arrange a pattern, such as a checker board, with regular contrast on a surface of the dewar 2. With this configuration, it is possible to measure a curved surface structure of the dewar 2 from the captured images with high accuracy, so that it is possible to determine the positional relationship among the image acquisition devices 5 and the dewar 2 with high accuracy.

A method of determining the positional relationship among the dewar 2 and the magnetic sensors 1 will be described below. The magnetic sensors 1 are incorporated in the dewar 2 at the time of shipment from a factory, and a positional relationship among the magnetic sensors 1 is known, for example. In other words, the positional relationship determination unit 62 is able to determine the positional relationship among the dewar 2 and the magnetic sensors 1 by referring to the known positional relationship that is stored in the memory device 25, for example.

Through the process as described above, the positional relationship determination unit 62 is able to determine the positional relationship among the head of the subject 10 and the magnetic sensors 1.

Here, even if the position of the head of the subject 10 is changed, the positional relationship among the image acquisition devices 5, the dewar 2, and the magnetic sensors 1 is unchanged. Therefore, the positional relationship determination unit 62 is able to calculate a three-dimensional change in the position of the head from amounts of change in the images of the head of the subject 10 that are captured again by the image acquisition devices 5. Further, the positional relationship determination unit 62 is able to determine a positional relationship among the newly-calculated position of the head and the magnetic sensors 1.

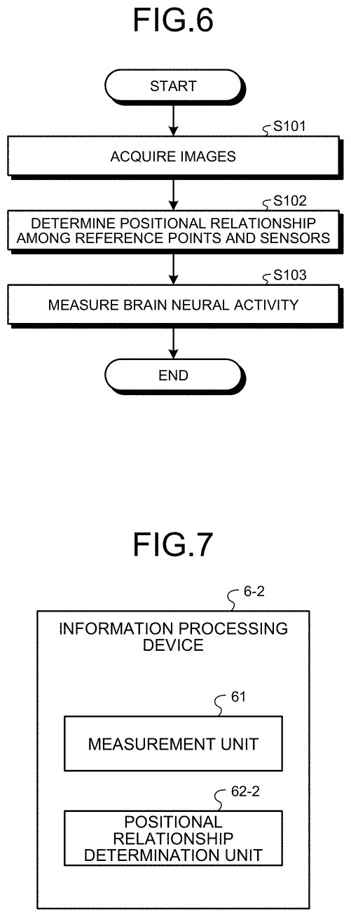

A flow of a positional relationship determination process according to the present embodiment will be described below. FIG. 6 is a flowchart illustrating an example of the flow of the positional relationship determination process according to the present embodiment.

The positional relationship determination unit 62 acquires images captured by the image acquisition devices 5a and 5b (Step S101). The positional relationship determination unit 62 determines the positional relationship among the reference points on the head of the subject 10 and the magnetic sensors 1 on the basis of the acquired images (Step S102). Thereafter, the measurement unit 61 measures brain neural activity on the basis of a biological signal detected by the magnetic sensors 1 (Step S103).

The case in which the two image acquisition devices 5 are provided has been described above. Even if three or more image acquisition devices are provided, it is possible to determine the positional relationship among the head of the subject 10 and the magnetic sensors 1 with high accuracy through the same process.

As described above, according to the first embodiment, a plurality of (N) image acquisition devices that are arranged such that image capturing directions are separated by a certain angle or more so as to capture a large number of reference points are used. With this configuration, it is possible to determine the positional relationship among the head of the subject and the sensors with high accuracy and improve reliability of analysis of biological information with respect to various positions and orientations of the head of the subject.

### Second Embodiment

In the first embodiment, the positional relationship among the head of the subject 10 and the magnetic sensors 1 is determined by capturing images of the head of the subject 10 and the dewar 2. The positional relationship among the image acquisition devices 5 and the magnetic sensors 1 may be determined by calibration that is performed in advance. A biological information measurement system according to a second embodiment includes a calibration process as described above.

A system configuration example of the biological information measurement system according to the second embodiment is the same as the system configuration of the first embodiment as illustrated in FIG. 1, and therefore, explanation thereof will be omitted. The second embodiment is different from the first embodiment in that the information processing device has a calibration function.

FIG. 7 is a diagram for explaining functions of an information processing device 6-2 according to the second embodiment. As illustrated in FIG. 7, the information processing device 6-2 includes the measurement unit 61 and a positional relationship determination unit 62-2. The function as the measurement unit 61 is the same as that of the first embodiment; therefore, the function is denoted by the same reference symbol and explanation thereof will be omitted.

The positional relationship determination unit 62-2 is different from the positional relationship determination unit 62 of the first embodiment in that the positional relationship determination unit 62-2 has a function (calibration function) to determine the positional relationship among the image acquisition devices 5 and the magnetic sensors 1 in advance.

In the calibration function, for example, an object, such as a mannequin, that has a known shape and that wears magnetic marker coils is used instead of the subject 10. The positional relationship determination unit 62-2 determines the positional relationship among the image acquisition devices 5 and the magnetic sensors 1 on the basis of images of the object that are captured by the image acquisition devices 5 and on the basis of magnetic data that represents magnetic fields generated by the magnetic marker coils and that are detected by the magnetic sensors 1.

Then, the positional relationship determination unit 62-2 is able to determine the positional relationship among the image acquisition devices 5 and the head of the subject 10 from the images of the head of the subject 10 that are captured again by the image acquisition devices 5, and is able to make an association with the positions of the magnetic sensors 1.

With this configuration, it is possible to determine the positional relationship among the head of the subject 10 and the magnetic sensors 1 in a non-contact manner without attaching the magnetic marker coils on the head of the subject 10 during measurement of magnetoencephalography. Further, in the calibration, it is possible to use the magnetic data in addition to image information, so that it is possible to determine the positional relationship with higher accuracy.

A method of arranging the plurality of image acquisition devices 5, which is applicable to each of the embodiments (the first embodiment and the second embodiment), will be described in detail below.

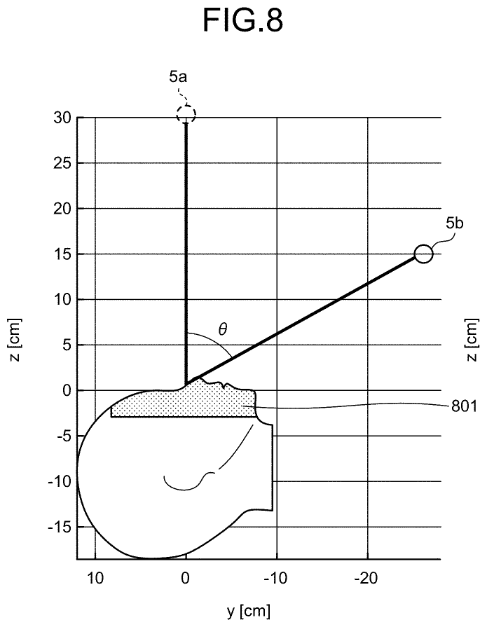

FIG. 8 to FIG. 10 are diagrams for explaining an example of the method of arranging the two image acquisition devices 5a and 5b. Similarly to FIG. 5, it is assumed that the image acquisition devices 5a and 5b capture the image 511a and the image 511b, respectively.

Each of the image 511a and the image 511b includes the reference points 51 on the surface of the three-dimensional head data. With an increase in the numbers of the different reference points 51 in the image 511a and the image 511b, it becomes possible to more accurately determine the positional relationship among the head of the subject 10 and the magnetic sensors 1. In other words, it is desirable to arrange the image acquisition devices 5a and 5b such that each of the image 511a and the image 511b include as many different reference points 51 as possible.

For example, the three-dimensional head data is divided into four regions as described below in the coordinate system of the three-dimensional head data.
A region RA including the reference points 51 that are captured in only the image 511a
A region RB including the reference points 51 that are captured in only the image 511b
A common region RC including the reference points 51 that are common to both of the image 511a and the image 511b
A region RD that is not captured in any of the image 511a and the image 511b

In the following, the respective numbers of three-dimensional head data points included in the above-described four regions are denoted by NA, NB, NC, and ND.

As described above, with an increase in the numbers of the different reference points 51 in the image 511a and the image 511b, it becomes possible to more accurately determine the positional relationship among the head of the subject 10 and the magnetic sensors 1. Therefore, it is preferable to increase (NA+NB) as much as possible and decrease NC as much as possible.

An example of a simulation for obtaining a preferable arrangement angle will be described below with reference to FIG. 8 to FIG. 10.

It is assumed that an angle of view of each of the image acquisition devices 5a and 5b is 15 degrees, and a distance between each of the image acquisition devices 5a and 5b and the subject 10 is 30 centimeters (cm). It is assumed that an interval between the reference points 51 is 3 millimeters (mm). It is assumed that an angle between the image capturing direction of the image acquisition device 5a and the image capturing direction of the image acquisition device 5b is denoted by θ. Here, for convenience of explanation, a case in which θ=60 degrees is illustrated.

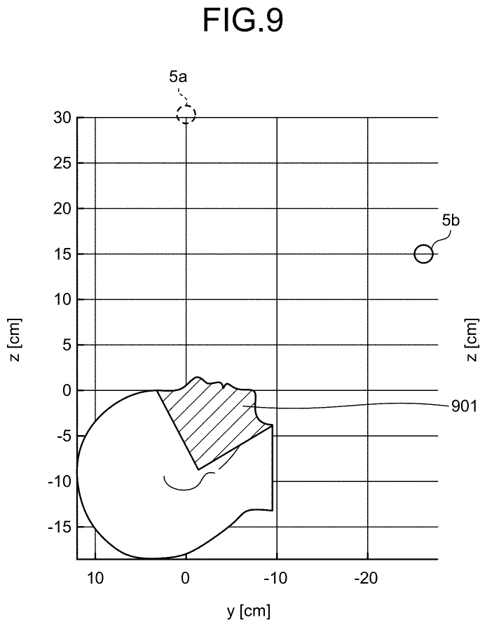

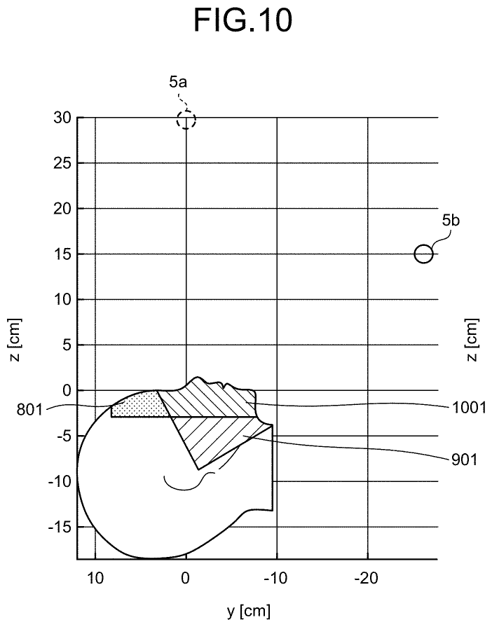

FIG. 8 to FIG. 10 are diagrams illustrating a relationship among the head of the subject 10 and reference point groups that are arranged on a head surface and that are captured by the image acquisition devices 5a and 5b. FIG. 8 is a diagram illustrating a relationship between the head of the subject 10 and a reference point group 801 that is arranged on the head surface and that is captured by the image acquisition device 5a. FIG. 9 is a diagram illustrating a relationship between the head of the subject 10 and a reference point group 901 that is arranged on the head surface and that is captured by the image acquisition device 5b. FIG. 10 is a diagram illustrating a relationship between the head of the subject 10 and a reference point group 1001 that is arranged on the head surface and that is captured by both of the image acquisition devices 5a and 5b.

As described above, the three-dimensional head data is divided into four regions as listed below.
The region RA (including the reference point group 801) that is captured by only the image acquisition device 5a
The region RB (including the reference point group 901) that is captured by only the image acquisition device 5b
The region RC (including the reference point group 1001) that is captured by the image acquisition device 5a and the image acquisition device 5b
The region RD (including a point group other than the reference point groups 801 and 901) that is not captured by any of the image acquisition device 5a and the image acquisition device 5b

Here, at each of θ=0, 10, 20, 30, 40, 50, 60, 70, 80, and 90 as the angle θ, the number of the reference points of the three-dimensional head data included in each of the regions RA, RB, RC, and RD is counted, and (NA+NB) and NC are calculated.

A range of θ>90 is eliminated from the simulation because the positions of the image acquisition devices 5 and the subject 10 interfere with one another.

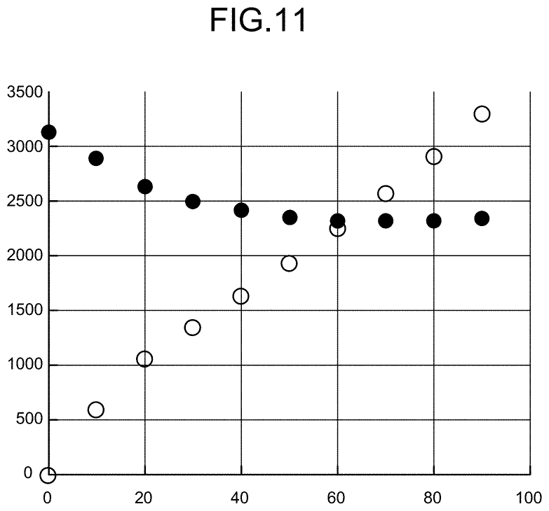

FIG. 11 is a graph illustrating an example of the calculated values. Specifically, FIG. 11 is a diagram illustrating a relationship between 8 (horizontal axis) and (NA+NB) and NC (vertical axis). White circles represent (NA+NB). Black circles represent NC.

(NA+NB) is a sum of the number NA of the reference points that are captured by only the image acquisition device 5a and the number NB of the reference points that are captured by only the image acquisition device 5b. Therefore, (NA+NB) tends to increase with an increase in θ. NC is the number of the reference points that are captured by both the two image acquisition devices 5a and 5b. Therefore, NC tends to decrease with an increase in θ.

By dividing the data into the four regions and counting the numbers of the reference points as described above, it is possible to determine an optimal angle (image capturing directions) of the image acquisition devices 5 by taking into account a balance among values of the reference points among the regions.

An upper limit of θ will be described below. The value of (NA+NB) becomes larger than the value of NC with an increase in θ. It is possible to determine the positional relationship among the head of the subject 10 and the magnetic sensors 1 with higher accuracy with an increase in the numbers of the different reference points in the image 511a and the image 511b; therefore, it is preferable that θ is larger. However, in the range of θ>90, the positions of the image acquisition devices 5 and the subject 10 interfere with one another. Therefore, it is preferable that θ is at least equal to or smaller than 90 degrees.

A lower limit of θ will be described below. First, θ=0 is inappropriate because the condition in which the two image acquisition devices 5a and 5b are included is not met. If 0<θ<90, a larger θ is more preferable. In contrast, as illustrated in FIG. 11, even if θ=10, (NA+NB)>0, and the reference points that are captured by only each of the image acquisition devices 5 are present.

In other words, it is possible to acquire information on the different reference points, and therefore, it is preferable that θ>0, and it is acceptable that θ=10, as long as the plurality of image acquisition devices do not physically interfere with one another.

The number of the reference points is set such that, for example, the value of (NA+NB) is larger than a first threshold and the value of NC is larger than a second threshold (a value larger than the first threshold). For example, if an interval between the reference points is set to 3 mm, as illustrated in FIG. 11, it is preferable that a condition in which (NA+NB)>500 (one example of the first threshold) and NC>2000 (one example of the second threshold) is met.

A method of setting the reference points is not limited to the method of setting the interval to 3 mm. By further reducing the interval between the reference points, it is possible to determine the positional relationship among the head of the subject 10 and the magnetic sensors 1 with higher accuracy. Even in this case, the condition in which (NA+NB)>500 and NC>2000 is met.

An example of setting more preferable e will be described below. As illustrated in FIG. 11, if θ=60, the value of (NA+NB) and the value of NC are approximately the same and the reference points on the head surface are captured with a good balance. Therefore, it is more preferable that θ=60.

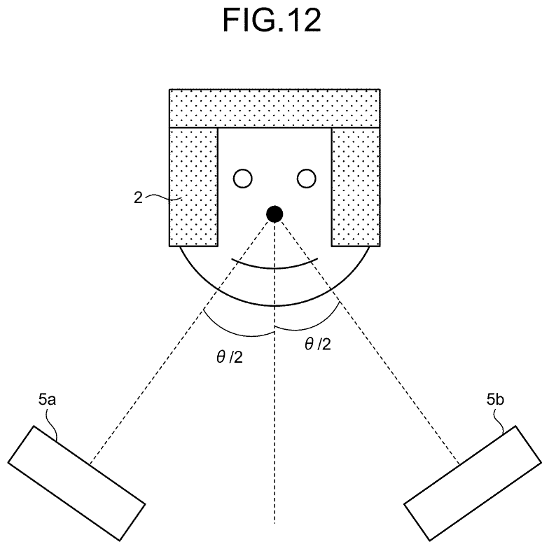

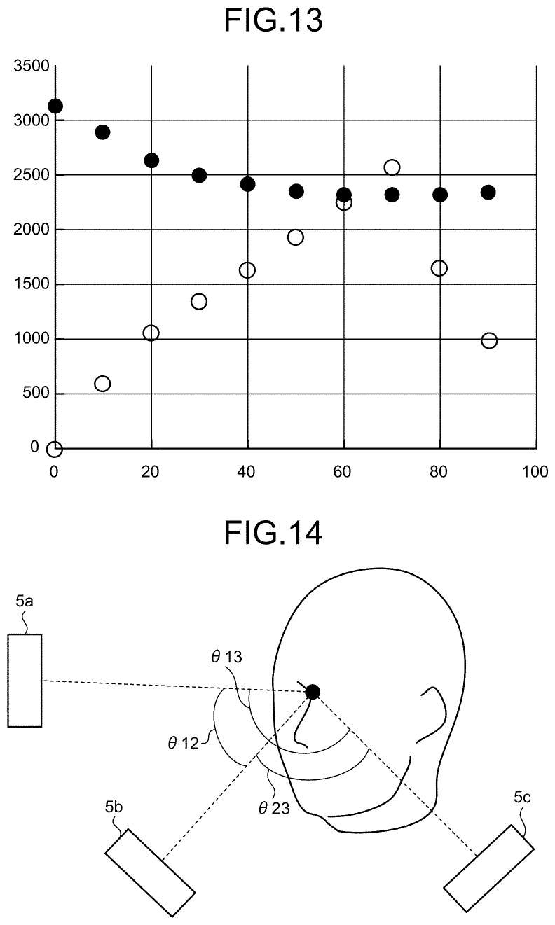

FIG. 8 to FIG. 11 illustrate the example in which any one of the image acquisition devices 5 (the image acquisition device 5a in the example in the drawings) is arranged in front of the subject 10, but an arrangement method is not limited to this example. FIG. 12 and FIG. 13 are diagrams for explaining another example of the method of arranging the image acquisition devices 5a and 5b.

As illustrated in FIG. 12, the image acquisition devices 5a and 5b may be arranged at the same angle (θ/2) with respect to the front of the subject 10. However, even in the range of 0<θ<90, if θ is extremely increased, the reference points on the surface of the head of the subject 10 may be hidden by the dewar 2 when viewed from the image acquisition devices 5. In this case, the number of the reference points that are captured by only the image acquisition device 5a or the image acquisition device 5b is reduced as compared to a case in which the reference points are not hidden. FIG. 13 is a diagram schematically illustrating the above-described situation, that is, a situation in which (NA+NB) decreases when e is increased.

If the situation as described above occurs, it is preferable to select certain θ (θ=70 in the example in FIG. 13) at which (NA+NB) is maximum in the range of 0<θ<90.

FIG. 14 is a diagram for explaining the method of arranging the three image acquisition devices 5. FIG. 14 illustrates an example of the method of arranging the image acquisition devices 5a and 5b and an image acquisition device 5c. If the three or more image acquisition devices 5 are arranged, it is preferable that an angle formed by the image capturing directions of the two image acquisition devices 5 that are arbitrarily selected from among all of the image acquisition devices 5 falls in a range of at least 60 to 90 degrees. In the example in FIG. 14, it is preferable that each of the three angles below falls in the range of 60 to 90 degrees.
An angle θ12 between the image capturing direction of the image acquisition device 5a and the image capturing direction of the image acquisition device 5b
An angle θ13 between the image capturing direction of the image acquisition device 5a and the image capturing direction of the image acquisition device 5c
An angle θ23 between the image capturing direction of the image acquisition device 5b and the image capturing direction of the image acquisition device 5c

As described above, the image acquisition devices 5 are not limited to monocular cameras, but may be stereo cameras. In this case, it is possible to determine a distance in a depth direction with high accuracy from information on disparity between two images that are captured by two lenses included in a single stereo camera. Therefore, it is possible to determine the positional relationship among the head of the subject 10 and the magnetic sensors 1 with high accuracy.

According to an embodiment, it is possible to determine a positional relationship among a head of a subject and sensors with high accuracy, and it is possible to improve reliability of analysis of biological information.

The above-described embodiments are illustrative and do not limit the present invention. Thus, numerous additional modifications and variations are possible in light of the above teachings. For example, at least one element of different illustrative and exemplary embodiments herein may be combined with each other or substituted for each other within the scope of this disclosure and appended claims. Further, features of components of the embodiments, such as the number, the position, and the shape are not limited the embodiments and thus may be preferably set. It is therefore to be understood that within the scope of the appended claims, the disclosure of the present invention may be practiced otherwise than as specifically described herein.

The method steps, processes, or operations described herein are not to be construed as necessarily requiring their performance in the particular order discussed or illustrated, unless specifically identified as an order of performance or clearly identified through the context. It is also to be understood that additional or alternative steps may be employed.

Further, any of the above-described apparatus, devices or units can be implemented as a hardware apparatus, such as a special-purpose circuit or device, or as a hardware/software combination, such as a processor executing a software program.

Further, as described above, any one of the above-described and other methods of the present invention may be embodied in the form of a computer program stored in any kind of storage medium. Examples of storage mediums include, but are not limited to, flexible disk, hard disk, optical discs, magneto-optical discs, magnetic tapes, nonvolatile memory, semiconductor memory, read-only-memory (ROM), etc.

Alternatively, any one of the above-described and other methods of the present invention may be implemented by an application specific integrated circuit (ASIC), a digital signal processor (DSP) or a field programmable gate array (FPGA), prepared by interconnecting an appropriate network of conventional component circuits or by a combination thereof with one or more conventional general purpose microprocessors or signal processors programmed accordingly.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), digital signal processor (DSP), field programmable gate array (FPGA) and conventional circuit components arranged to perform the recited functions.

What is claimed is:

1. A biological information measurement system comprising:
a cover member configured to cover a head of a subject, a plurality of sensors configured to detect a biological signal being arranged in the cover member;
a measurement unit configured to measure brain neural activity, based on the biological signal detected by the plurality of sensors;
N image capturing units configured to each acquire an image including at least three reference points and the cover member, the at least three reference points being set in relation to the subject, N being an integer equal to or larger than two; and
a positional relationship determination unit configure to determine a positional relationship among a plurality of reference points on the subject and the plurality of sensors, based on positional relationship data among the plurality of reference points and the cover member, the positional relationship data being obtained based on N pieces of image data obtained from the image capturing units, wherein
an angle between image capturing directions of two image capturing units among the N image capturing units is larger than zero degree and smaller than 90 degrees.

2. The biological information measurement system according to claim 1, wherein arrangement positions and the image capturing directions of the two image capturing units among the N image capturing units are set such that a sum of numbers each being a number of reference points included in only an image captured by one of the two image capturing units is larger and a number of reference points included in both two images captured by the two image capturing units is smaller.

3. The biological information measurement system according to claim 2, wherein the arrangement positions and the image capturing directions of the two image capturing units among the N image capturing units are set such that the sum is larger than a first threshold and the number of the reference points included in both the two images captured by the two image capturing units is larger than a second threshold that is larger than the first threshold.

4. The biological information measurement system according to claim 3, wherein the first threshold is 500, and the second threshold is 2000.

5. The biological information measurement system according to claim 1, wherein the angle is larger than 60 degrees and smaller than 90 degrees.

6. The biological information measurement system according to claim 1, wherein arrangement positions and the image capturing directions of the two image capturing units among the N image capturing units are determined such that a sum of numbers each being a number of reference points included in only an image captured by one of the two image capturing units is maximized.

7. The biological information measurement system according to claim 1, wherein the image capturing units comprises stereo cameras.

8. The biological information measurement system according to claim 1, wherein the plurality of sensors comprise magnetic sensors.

9. A non-transitory computer-readable medium including programmed instructions that cause a computer configured to control a biological information measurement system including: a cover member configured to cover a head of a subject, a plurality of sensors for detecting a biological signal being arranged in the cover member; a measurement unit configured to measure brain neural activity, based on the biological signal detected by the plurality of sensors; and N image capturing units configured to each capture an image including at least three reference points and the cover member, the at least three reference points being set in relation to the subject, N being an integer equal to or larger than two, to function as:
a positional relationship determination unit configured to determine a positional relationship among a plurality of reference points on the subject and the plurality of sensors, based on positional relationship data among the plurality of reference points and the cover member, wherein
an angle between image capturing directions of two image capturing units among the N image capturing units is larger than zero degree and smaller than 90 degrees.
